(19) United States
(12) Patent Application Publication
(10) Pub. No.: US 2017/0046582 A1
(43) Pub. Date: Feb. 16, 2017

(54) DRIVER MONITOR DEVICE

(71) Applicant: DENSO CORPORATION, Kariya-city, Aichi-pref. (JP)

(72) Inventor: Kazumichi HOSHIYA, Kariya-city (JP)

(21) Appl. No.: 15/306,596

(22) PCT Filed: Jul. 10, 2015
(86) PCT No.: PCT/JP2015/003511

§ 371 Date: Oct. 25, 2016

(30) Foreign Application Priority Data
Jul. 30, 2014 (JP) 2014-154850

(51) Int. Cl. G06K 9/00; B60Q 3/02; A61B 5/18; G03B 15/03; H04N 5/225; H04N 5/33

(52) U.S. Cl. CPC G06K 9/00845; H04N 5/2256; H04N 5/33; A61B 5/18; G03B 15/03; B60Q 3/024; B60Q 3/0293

(57) ABSTRACT

A driver monitor device includes: multiple light sources for irradiating light toward a driver seat respectively; a shooting unit arranged at a position from which the shooting unit shoots a driver sitting on the driver seat; a driver detection unit detecting a position of a face or an eye of the driver based on information in an image shot by the shooting unit; a light source specifying unit specifying one of the light sources which irradiates an area of the face or the eye of the driver, according to a detection result of the driver detection unit; and a light source control unit controlling the light sources to turn on the one of the light sources specified by the light source specifying unit, and to turn off or dim another light source.

### CROSS REFERENCE TO RELATED APPLICATION

This application is based on Japanese Patent Application No. 2014-154850 filed on Jul. 30, 2014, the disclosure of which is incorporated herein by reference.

### TECHNICAL FIELD

The present disclosure relates to a driver monitor device mounted on a vehicle.

### BACKGROUND ART

Conventionally, a driver monitor device is well known that the device shoots a face of a driver, detects a direction of the face and an opening degree of an eye using an image recognition process, and gives an alert or warning to the driver (for example, in Patent Literature 1). In the driver monitor device, various techniques are made such that light emitted from a near infra-red light LED is expanded radially by a lens so as to irradiate a whole of the face of the driver when the near infra-red light LED is used as a light for irradiating the face of the driver.

However, the face of the driver is not always disposed at the same position. When the whole of the face of the driver is always irradiated, it is necessary to expand the light from the near infra-red light LED to a wide area so as to irradiate throughout a possible range in which the face of the driver is disposed. As a result, since the light always irradiates an area in which the face of the driver is not disposed, an unnecessary electric power is wasted.

### PRIOR ART LITERATURES

### Patent Literature

Patent Literature 1: JP-2013-146032-A

### SUMMARY OF INVENTION

It is an object of the present disclosure to provide a driver monitor device with electric power consumption which is restricted.

According to an aspect of the present disclosure, a driver monitor device includes: a plurality of light sources each of which irradiates light toward a driver seat; a shooting unit that is arranged at a position from which the shooting unit shoots a driver sitting down on the driver seat; a driver detection unit that detects a position of a face or a position of an eye of the driver based on information in an image shot by the shooting unit; a light source specifying unit that specifies one of the light sources which irradiates an area of the face or the eye of the driver, according to a detection result of the driver detection unit; and a light source control unit that controls the plurality of light sources to turn on the one of the light sources specified by the light source specifying unit, and to turn off or dim another light source.

According to the above features, the device turns off or dims another light source that does not irradiate an area of the face or the eye of the driver in a possible region in which the face of the driver is disposed, so that the light source does not irradiate brightly an area which is not necessary in case of monitoring the driver. Thus, the electric power consumed by the driver monitor device is restricted to be low.

### BRIEF DESCRIPTION OF DRAWINGS

The above and other objects, features and advantages of the present disclosure will become more apparent from the following detailed description made with reference to the accompanying drawings. In the drawings:

FIG. 1 is a diagram showing a cross sectional schematic view of a vehicle indicative of a mounting position of a driver monitor device according to a first embodiment;

FIG. 2 is a diagram showing a perspective view indicative of an overview of the driver monitor device according to the first embodiment;

FIG. 3 is a diagram showing a constitution of a controller in the driver monitor device and a relationship between a light source and a face of a driver specified by a light source specifying unit according to the first embodiment;

FIG. 4 is a flowchart showing an example of a control executed by the controller according to the first embodiment; and

FIG. 5 is a diagram showing an arrangement position of the light source according to a second embodiment.

### EMBODIMENTS FOR CARRYING OUT INVENTION

Embodiments of the present disclosure will be explained with reference to drawings as follows. Here, in each embodiment, a corresponding element has the same reference number, and duplicated explanation may be skipped. When only a part of constitutions in each embodiment is explained, other parts of the constitutions may be applied to constitutions previously explained in other embodiments. Further, in addition to a combination of constitutions described in each embodiment, constitutions in multiple embodiments may be combined partially although a combination is not clearly described as long as the combination does not include inconsistency.

### First Embodiment

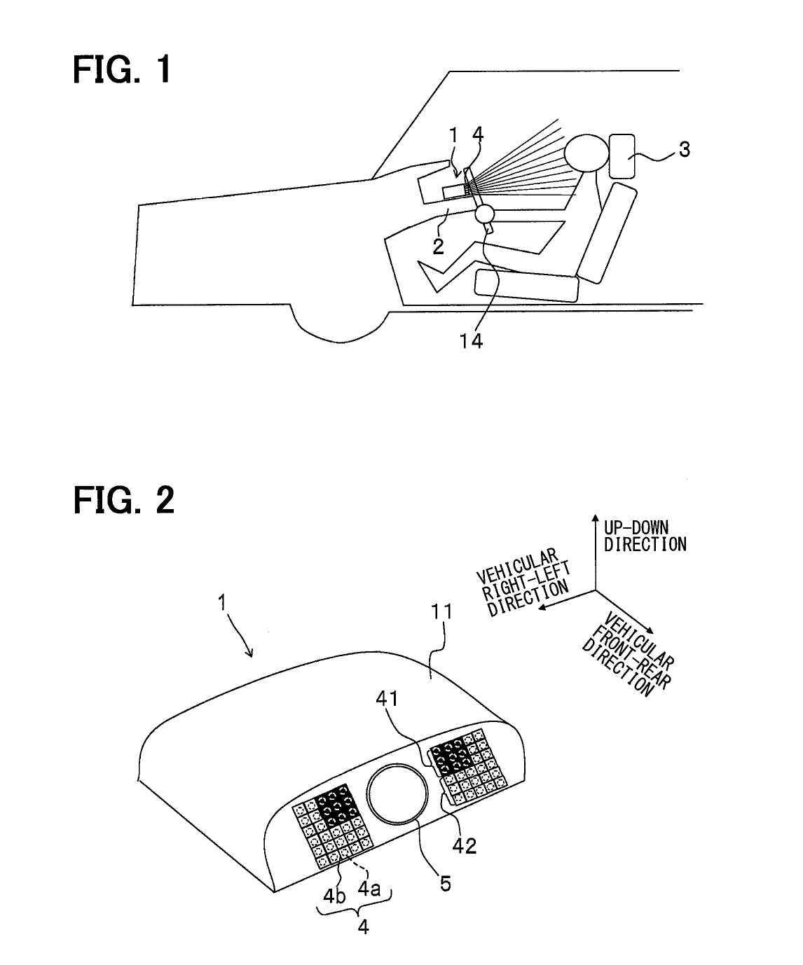

FIG. 1 is a diagram showing a cross sectional schematic view of a vehicle indicative of a mounting position of a driver monitor device according to the present embodiment. The driver monitor device 1 according to the present embodiment has a case 11 mounted on a steering wheel column 2. Multiple LED light sources 4 for emitting light respectively toward a driver seat 3 are arranged on a periphery of a driver seat side of the case 11.

FIG. 2 is a diagram showing a perspective view indicative of an overview of the case 11 in the driver monitor device 1 according to the first embodiment. Above described multiple LED light sources 4 are arranged in a concentrated manner at two places on right and left sides of the case 11 respectively on side peripheries of the driver seat to sandwich a near infra-red light camera 5. The near infra-red light camera 5 is a shooting unit arranged at a position capable of shooting an upper body of the driver through a clearance between spokes of a steering wheel 14. Multiple LED light sources 4 include multiple LEDs 4a as a light emitting unit and multiple lenses 4b arranged in front of each LED 4a. Here, each lens 4b according to the present embodiment has an almost square shape in a front view. The lenses 4b are arranged in a matrix manner having multiple rows and multiple columns. The LED 4a according to the present embodiment is arranged on a back of the lens 4b, and emits irradiation light having high directivity using the lens 4b toward the driver.

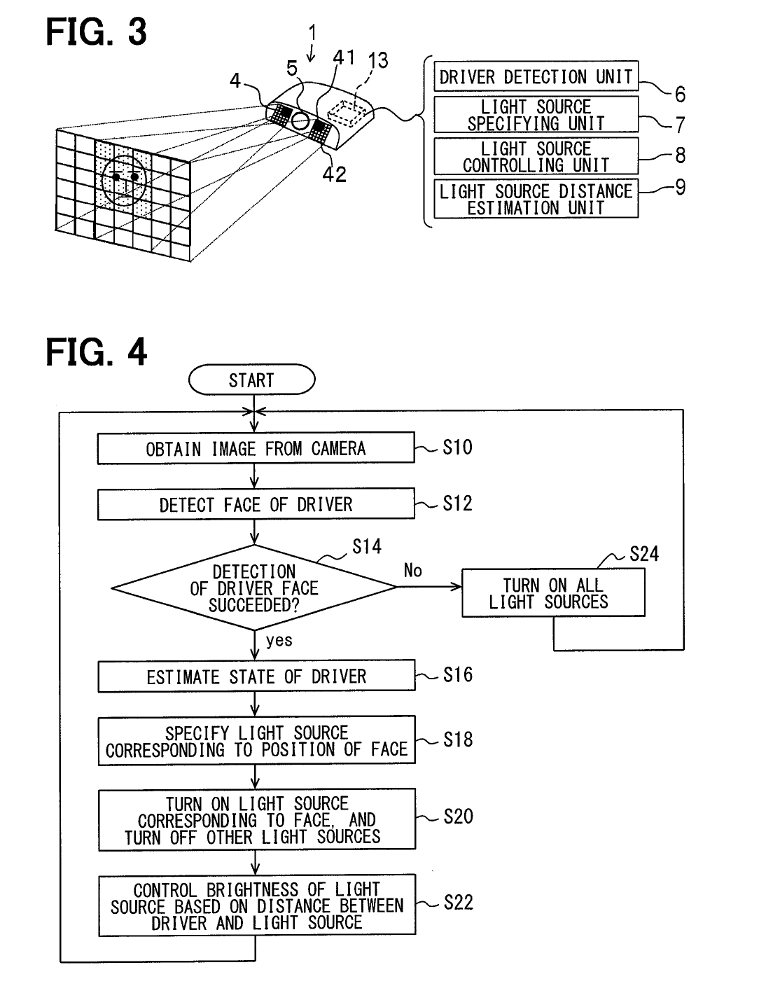

FIG. 3 is a diagram showing a constitution of the driver monitor device 1 and a relationship between a light source group 41 and a position of a face of the driver specified by a light source specifying unit 7 according to the first embodiment. A controller 13 of the driver monitor device 1 is accommodated in the case 11. The controller 13 includes a not-shown calculation device and a not-shown memory, which provide: a driver detection unit 6 that detects a position of the face of the driver or the position of the eye of the driver based on an image obtained from the near infra-red light camera 5; a light source specifying unit 7 that specifies a LED light source group 41 capable of irradiating an area of the face or the eye of the driver among above described multiple LED light sources 4 based on a detection result of the driver detection unit 6; a light source control unit 8 that controls light sources to turn on the LED light source group 41 specified by the light source specifying unit 7 to turn off or to dim the other light source group 42; and a light source distance estimation unit 9 that detects a distance between the driver and the LED light source group 41.

Further, as shown in FIG. 3, the LED light sources 4 are arranged and concentrated in a matrix manner and divided into right and left parts on the periphery of the case 11 on the driver seat side, and the sources 4 irradiate and share a right side area and a left side area including the face of the driver. When the driver sits down on the driver seat with a correct posture, an upper central part of the LED light sources 4 in the matrix manner divided into right and left sides is the LED light source group 41 capable of irradiating the area of the face or the eye of the driver, and the other part of the sources 4 is the other LED light source group 42 which is turned off or dimmed.

Next, a process executed by the controller 13 according to the present embodiment will be explained with reference to the flowchart in FIG. 4. At first, at step S10, the controller 13 obtains the image shot a near area of the driver seat to include the face of the driver from the near infra-red light camera 5. Then, the controller 13 proceeds to step S12.

At step S12, the controller 13 detects the position of the face or the position of the eye of the driver based on the image obtained from the near infra-red light camera 5. Then, the controller 13 proceeds to step S14.

At step S14, the controller 13 determines whether the position of the face or the position of the eye of the driver is detected at step S12. When the controller 13 determines that the position of the face or the position of the eye is detected, the controller 13 proceeds to step S16.

At step S16, the controller 13 estimates the state of the driver such as a face direction, the opening degree of the eyelid, the sleepiness, and the degree of absent-mindedness of the driver according to the position of the face or the position of the eye detected at step S14. If necessary, a not-shown warning device gives an warning or a stimulus to the driver. After the estimation of the driver consciousness and the warning or the stimulus to the driver are completed at step S16, the controller 13 proceeds to step S18.

At step S18, the LED group 41 capable of emitting the near infra-red light toward the position of the face or the position of the eye detected at step S14 is specified from the above described multiple LEDs 4. Then, it goes to step S20. Specifically, it is determined which position of the irradiation areas of multiple LEDs 4 correspond to the position of the face or the position of the eye detected at step S14. More specifically, the detail will be explained as an example in FIG. 3. When the camera 5 catches the face of the driver at a slightly upper area of the front face of the camera 5, the LED group 41 arranged on an upper central area of the case 11 is specified as the LED group capable of emitting the near infra-red light from each of two group of the LED light sources 4 arranged on right and left sides of the camera 5.

At step S20, the LED light source group 41 specified at step S18 by the light source specifying unit 7 is turned on, and the other light source group 42 is turned off or dimmed. Then, it goes to step S22.

At step S22, the distance between the LED light source 4 and the driver is estimated from the shot image of the driver, and the output of the LED light source 4 is adjusted according to the estimated distance between the LED light source 4 and the driver. Then, it returns to step S10. Specifically, when the distance between the LED light source 4 and the driver is short, the light from the LED light source 4 may reach the driver with high intensity. Thus, it is preferable to adjust such that, the shorter the distance between the LED light source 4 and the driver, the smaller the output of the LED light source 4.

On the other hand, when it is determined at step S14 that the position of the face or the position of the eye is not detected, it goes to step S24. At step S24, all of the LED light sources 4 are turned on, and it returns to step S10. Then, the image is obtained under a condition that a wide area is irradiated by the LED light sources 4, and the face or the eye of the driver is searched from the image.

Here, when the driver monitor device 1 is activated after the accessory electric power source of the vehicle is turned on, and the device 1 firstly executes the process in FIG. 4, at step S10 at the first time, the image shot by the camera 5 is obtained under a condition that the LED light sources 4 are not turned on. Thus, in many cases, the detection of the image of the face or the eye at step S12 may be failed, and the determination at step S14 is negative. Then, it goes to step S24. The process in FIG. 4 is repeatedly executed, and therefore, in the process at the second time or more, the LED light sources 4 are turned on. Thus, it does not matter.

According to the above features, the LED light sources 41 which do not irradiate the area of the face of the driver are turned off, or dimmed as shown in FIG. 3. Thus, an area not necessary in case of monitoring the driver is not irradiated, and therefore, the electric power consumed by the driver monitor device is restricted. It is not necessary to target a whole of the area of the face of the driver. Alternatively, the position of the eye of the driver may be detected, the LED group 41 for irradiating the area of the eye of the driver may be specified, and the other LED group 42 may be turned off or dimmed.

### Second Embodiment

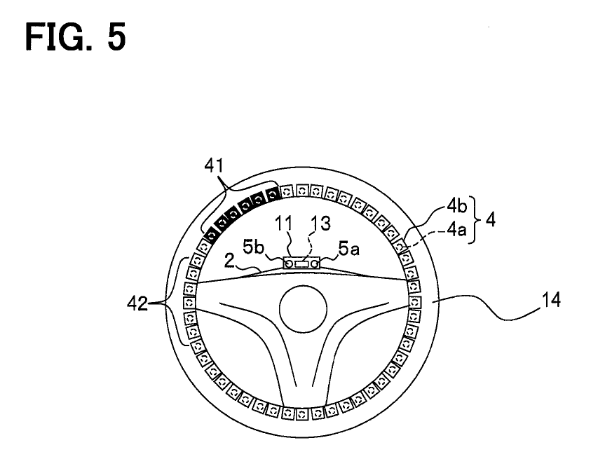

In the above described first embodiment, the case 11 of the driver monitor device 1 having multiple LED light sources 4 is arranged on the steering column 2. The present disclosure is not limited to this feature. As later described in the second embodiment, multiple LED light sources 4 may be arranged on an inside of the steering wheel 14. Specifically, as shown in FIG. 5, the lenses 4b are arranged on an inner circumference of the steering wheel in a ring manner. The LED 4a is disposed as a light source in each lens 4b.

In the present embodiment, it is necessary to determine the position of the LED light source group 41, for emitting light to the area corresponding to the position of the face or the position of the eye of the driver, according to the rotation position of the steering wheel 4. The light source specifying unit 8 in the controller 13 according to the present embodiment has an input parameter which is a rotation angle of the steering wheel.

Further, in the first embodiment, the monocular near infra-red light camera 5 is used to shoot an upper body of the driver who sits down on the driver seat. The distance between the driver and the light source is approximately estimated based on the size of the image of the driver shot by the camera. In the second embodiment, the shooting unit is a stereo camera having two near infra-red light cameras. Based on the stereoscopic parallax between two near infra-red light cameras 5a, 5b, the distance between the driver and the camera is estimated. Based on the positional relationship between the camera and the LED light source group 41 on the steering wheel 14 specified by the light source specifying unit 7, the distance between the driver and the LED light source 41 is obtained. According to the distance, the brightness of the LED light source 41 may be adjusted. Here, he positional relationship between the camera and the LED light source group 41 on the steering wheel 14 specified by the light source specifying unit 7 may be preliminary stored in a storage device.

Further, in the above described embodiment, the shooting unit is the near infra-red light camera 5. Alternatively, the shooting unit may be a visible light camera such as a CCD camera. Further, the LED light sources 4 having the LED 4a and the lens 4b are used as multiple light sources for irradiating the face of the driver. It is not always necessary to provide the lens 4b as a different body. Each LED light source 4 emits light having a high directivity, and each irradiation area is slightly overlapped each other and disposed at differently, so that multiple LEDs 4 assemble, and are preferably arranged so as to irradiate uniformly the upper body of the driver sitting down on the driver seat. In this case, when the LED light source emits light toward a separate position, it is not necessary to expand forcedly a small amount of the light from the LED light source in order to irradiate. Even when the light from each LED light source 4 is light having a weak brightness, the light is combined with the light from the neighboring LED light source 4 so that the light can irradiate an area which is far from the light source 4.

The above disclosure has the following aspects.

According to an aspect of the present disclosure, a driver monitor device includes: a plurality of light sources each of which irradiates light toward a driver seat; a shooting unit that is arranged at a position from which the shooting unit shoots a driver sitting down on the driver seat; a driver detection unit that detects a position of a face or a position of an eye of the driver based on information in an image shot by the shooting unit; a light source specifying unit that specifies one of the light sources which irradiates an area of the face or the eye of the driver, according to a detection result of the driver detection unit; and a light source control unit that controls the plurality of light sources to turn on the one of the light sources specified by the light source specifying unit, and to turn off or dim another light source.

According to the above features, the device turns off or dims another light source that does not irradiate an area of the face or the eye of the driver in a possible region in which the face of the driver is disposed, so that the light source does not irradiate brightly an area which is not necessary in case of monitoring the driver. Thus, the electric power consumed by the driver monitor device is restricted to be low.

Alternatively, the driver monitor device may further include: a distance detection unit that detects a distance between the one of the light sources and the driver. The light source control unit adjusts a brightness of the one of the light sources according to a detection result of the distance detection unit. Thus, when the distance between the driver and the light source is short, it is not necessary to brighten the light source in an undesirable manner, so that the electric power consumed by the driver monitor device is much reduced.

Alternatively, the plurality of the light sources may be mounted on a steering wheel in a ring manner. The light source specifying unit obtains rotation information of the steering wheel, and specifies the one of the light sources to turn on based on the rotation information of the steering wheel. Thus, the electric power consumption of the driver monitor device is reduced, and it is possible to minimize the case of the driver monitor device arranged on the steering column by mounting the plurality of light sources on the steering wheel.

It is noted that a flowchart or the processing of the flowchart in the present application includes sections (also referred to as steps), each of which is represented, for instance, as S10. Further, each section can be divided into several sub-sections while several sections can be combined into a single section. Furthermore, each of thus configured sections can be also referred to as a device, module, or means.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A driver monitor device comprising:
a plurality of light sources each of which irradiates light toward a driver seat;
a shooting unit that is arranged at a position from which the shooting unit shoots a driver sitting down on the driver seat;
a driver detection unit that detects a position of a face or a position of an eye of the driver based on information in an image shot by the shooting unit;
a light source specifying unit that specifies one of the light sources which irradiates an area of the face or the eye of the driver, according to a detection result of the driver detection unit; and
a light source control unit that controls the plurality of light sources to turn on the one of the light sources specified by the light source specifying unit, and to turn off or dim another light source.

2. The driver monitor device according to claim 1, further comprising:
a distance detection unit that detects a distance between the one of the light sources and the driver, wherein:
the light source control unit adjusts a brightness of the one of the light sources according to a detection result of the distance detection unit.

3. The driver monitor device according to claim 1, wherein:
the plurality of the light sources are mounted on a steering wheel in a ring manner; and
the light source specifying unit obtains rotation information of the steering wheel, and specifies the one of the light sources to turn on based on the rotation information of the steering wheel.
